(19) United States
(12) Patent Application Publication
(10) Pub. No.: US 2025/0288433 A1
(43) Pub. Date: Sep. 18, 2025

(54) Breast On-Plant Device

(71) Applicants: Oren Tepper, Old Greenwich, CT (US); Evan Garfein, New York, NY (US)

(72) Inventors: Oren Tepper, Old Greenwich, CT (US); Evan Garfein, New York, NY (US)

(21) Appl. No.: 19/080,291

(22) Filed: Mar. 14, 2025

Related U.S. Application Data
(60) Provisional application No. 63/565,646, filed on Mar. 15, 2024.

(51) Int. Cl. A61F 2/52; A61F 2/50; B29C 33/40; B29L 31/00

(52) U.S. Cl. CPC A61F 2/52; B29C 33/405; A61F 2002/5053; A61F 2240/007; B29L 2031/7532

(57) ABSTRACT

A mold, apparatus, and method for providing a realistic simulation of a breast augmentation. The mold is an onlay mold that is additively manufactured with a negative mold of a topography of a native breast on one side and an ideal augmented topography on the opposite side. The mold is adherable to a torso of a patient over a native breast to illustrate a real look and feel of a completed augmentation. The mold may also be positioned within a pocketed brassiere to provide the simulation. The mold is generated from a three dimensional image taken of the patient's torso and breast.

### CROSS-REFERENCE TO RELATED APPLICATION

The present application claims priority to, and the benefit of, U.S. Provisional Application No. 63/565,646, which was filed on Mar. 15, 2024, and is incorporated herein by reference in its entirety.

### FIELD OF THE INVENTION

The present invention generally relates to a device for simulating a breast implant. More specifically, the present invention relates to a breast onlay mold for simulating a surgical breast enhancement or reconstruction in the physical world and a method of simulating a breast augmentation. Accordingly, the present specification makes specific reference thereto. However, it is to be appreciated that aspects of the present invention are also equally amenable to other like applications, devices and methods of manufacture.

### BACKGROUND

Breast augmentation, or augmentation mammoplasty, is a surgical procedure performed to increase breast size and shape. A surgeon places breast implants under existing breast tissue or chest muscles to reshape the breasts. This procedure is done to enhance appearance, improve mental health, to correct anatomical deformity or asymmetry, for breast reconstruction, and the like.

When a woman contemplates augmentation of her breast by any means (fat injection, silicone implant placement), one of the most important questions she and her surgeon must answer is, how much larger would she like to be and what would this actually look and feel like? While there are limitations imposed by anatomy, biology, and sound surgical judgement on this question, the better the patient can imagine the effect of a given augmentation, the more likely she is to be satisfied with the operation.

Traditionally, surgeons have offered patients samples of implants of various sizes to “try on” by placing the implant over the breast and within a bra. More recently, surgical software has enabled surgeons to simulate on a computer screen the effect of an augmentation with a particular implant in a given patient with relatively high fidelity. The patient can see her own body with a simulated implant placed beneath the breast. There are even three dimensional models of the patient's torso with a simulated augmentation. All of these offer the patient and surgeon approximations of what she might look like. However, none of these options offer the patient the reality of what an augmentation might look and feel like on her own body prior to actually performing the surgery.

Accordingly, there is a great need for a way to simulate an augmented or reconstructed breast. There is also a great need for a breast onlay mold that is customizable to an existing human chest and breast geometry. There is also a great need for a breast onlay mold that can approximate a breast augmentation in both appearance and feel. Further, there is also a great need for a way to physically simulate the effect of an implant placement or fat injection to augment the breast prior to an augmentation surgery. Finally, there is a great need for a method of simulating a breast augmentation with a real-life application.

In this manner, the improved breast onlay mold of the present invention accomplishes all of the foregoing objectives, thereby providing an easy solution for non-invasively simulating an augmented or reconstructed breast. A primary feature of the present invention is a breast onlay mold designed to effectively conform to an existing human chest and breast geometry. Another feature of the present invention is a breast onlay mold which can approximate a breast augmentation in both appearance and feel. Further, the improved breast onlay mold of the present invention is capable of simulating the effect of an implant placement or fat injection to augment the breast prior to an augmentation surgery. Finally, the present invention includes a real-life simulation of a breast augmentation that feels and appears realistic.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a breast onlay mold. The breast onlay mold approximates a surface topography of an augmented breast. The breast onlay mold comprises a body. The body comprises an outward facing side and an inward facing side. The outward facing side is configured to approximate an ideal augmented topography. The outward facing side comprises a nipple extending from the outward facing side that may be customizable. The inward facing side comprises a negative mold of a native breast. The negative mold of the native breast is customized to fit a patient's existing chest wall and breast anatomy and is configured to approximate a surface topography of the native breast.

The breast onlay mold further comprises an adhesive layer. The adhesive layer is applied to the inward facing side of the body. The adhesive layer may be a silicone adhesive configured to temporarily secure the body over the native breast. The breast onlay mold may further comprise a release layer. The release layer temporarily covers and protects the adhesive layer prior to application. The breast onlay mold may further comprise a variety of skin tones or colors to mimic a realistic appearance.

The subject matter disclosed and claimed herein, in an additional embodiment thereof, comprises a breast augmentation simulating apparatus. The breast augmentation simulating apparatus comprises a brassiere and a pair of breast onlay molds. The brassiere comprises a pair of cups, each cup comprising a pocket. Each pocket is configured to retain one of the breast onlay molds and may comprise a negative mold of a native breast.

Each breast onlay mold comprises a body. Each body comprises an outward facing side and an inward facing side. Each outward facing side is configured to approximate an ideal augmented topography. Each outward facing side comprises a nipple extending from the outward facing side that may be customizable. Each inward facing side comprises a negative mold of a native breast. Each negative mold of the native breast is customized to a patient's existing chest wall and breast anatomy and is matches the respective negative mold of the respective pocket of the brassiere.

The subject matter disclosed and claimed herein, in an additional embodiment thereof, comprises a method of simulating a breast augmentation on a person. The method begins by obtaining a three dimensional image of the patient's upper front torso and breasts. Next, a breast onlay mold is additively manufactured or molded in a soft material. The breast onlay mold may be manufactured in customizable volumes, shapes, and weights. Once manufactured, the breast onlay mold is applied by skin adhesive or positioned in a special brassiere to the patient's chest provide the best possible approximation of augmentation, mimicking both the volume change and the effect it will have on the skin.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of a representation of a breast onlay mold of the present invention positioned on a human upper front torso area in accordance with the disclosed architecture;

FIG. 2 illustrates a perspective view of the breast onlay mold of the present invention positioned on a human upper front torso area in accordance with the disclosed architecture;

FIG. 3 illustrates a side view of the breast onlay mold of the present invention about to be positioned on a human upper front torso area in accordance with the disclosed architecture;

FIG. 4 illustrates a side view of the breast onlay mold of the present invention in accordance with the disclosed architecture;

FIG. 5 illustrates a side view of the breast onlay mold of the present invention positioned on a human upper front torso area in accordance with the disclosed architecture;

FIG. 6 illustrates a side view of the breast onlay mold of the present invention positioned on a human upper front torso area in accordance with the disclosed architecture;

FIG. 7 illustrates a rear view of a breast augmentation simulating apparatus of the present invention in accordance with the disclosed architecture;

FIG. 8 illustrates a side view of the breast augmentation simulating apparatus of the present invention in accordance with the disclosed architecture;

FIG. 9 illustrates a method of simulating a breast augmentation on a person of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, is a high fidelity tool for surgeons and patients designed to simulate the effect of an implant placement or fat injection to augment a human breast. This concept is applicable to cosmetic breast enhancement, partial breast reconstruction after lumpectomy, or total reconstruction of the breast after mastectomy for cancer. The tool is designed to create a simulated breast onlay mold based on the surface topography of the augmented or reconstructed breast in the physical world.

The breast onlay mold is a removable temporary mold that can be generated in different sizes and shapes. The breast onlay mold may be printed or created from different materials to make the breast firmer or softer. A nipple position on the breast onlay mold can be adjusted or customized. The breast onlay mold is configured to adhesively attach to the skin or may alternatively be fitted into a custom brassiere. The breast onlay mold is further configured to simulate the entire augmented breast volumetrically and topographically. The breast onlay mold is designed to create the most realistic approximation of an augmentation both in and out of clothing. The breast onlay mold is designed to create the most realistic approximation of the weight of an augmented breast.

The breast onlay mold is further designed to assist plastic surgeons and their patients who are considering cosmetic breast augmentation procedures. The breast onlay mold is also designed to provide consumer a temporary, anatomically realistic, non-surgical augmentation in much the same way that hair extensions or false eyelashes are utilized. Further, the breast onlay mold is also useful for assisting patients with visualizing reconstructive goals after breast cancer surgery.

The breast onlay mold is an onlay mold that is additively manufactured with a negative mold of a topography of a native breast on one side and an ideal augmented topography on the opposite side. The breast onlay mold is adherable to a torso of a patient over a native breast to illustrate a real look and feel of a completed augmentation, or may be positioned within a pocketed brassiere to provide the simulation. The breast onlay mold is generated from a three dimensional image taken of the patient's torso and breast.

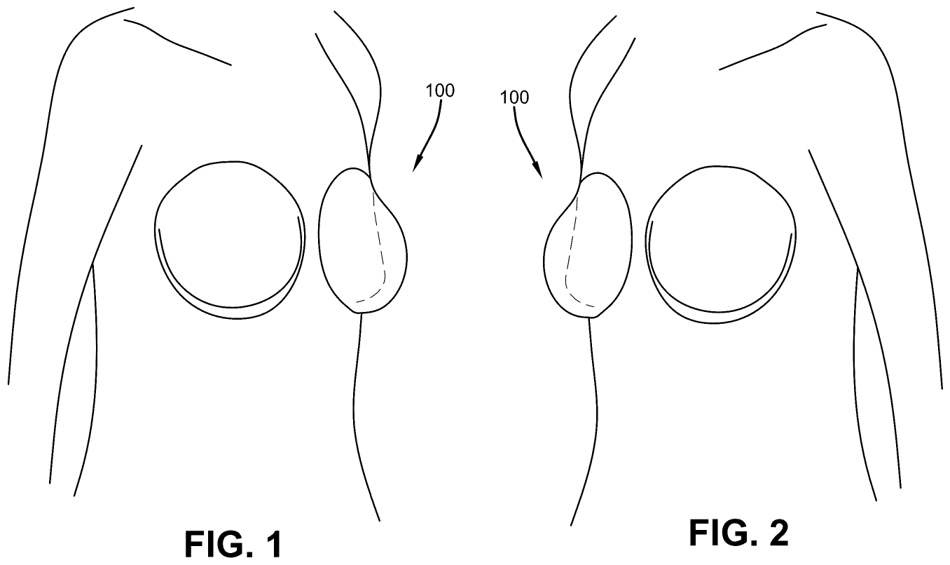

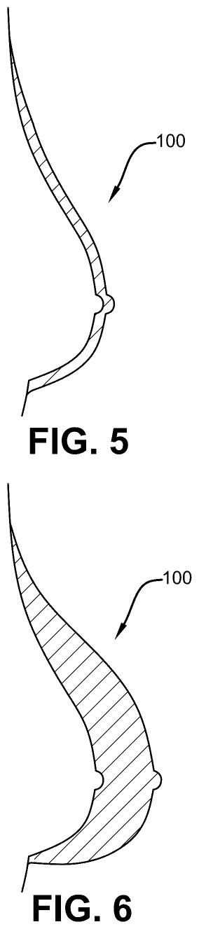

Referring initially to the drawings, FIGS. 1-6 illustrate a breast onlay mold 100. As illustrated in FIGS. 1 and 2, the breast onlay mold 100 is positional and temporarily secured over a native breast. The breast onlay mold 100 approximates a surface topography of an augmented breast in look and feel.

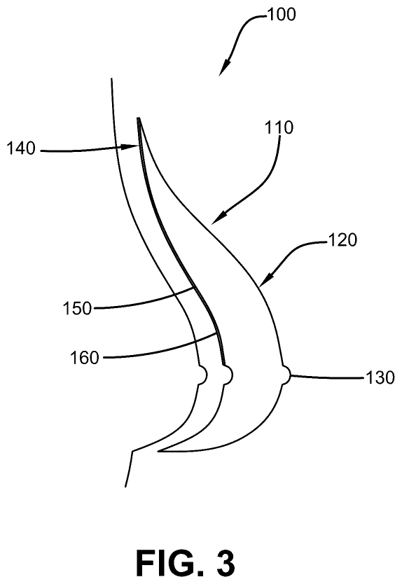

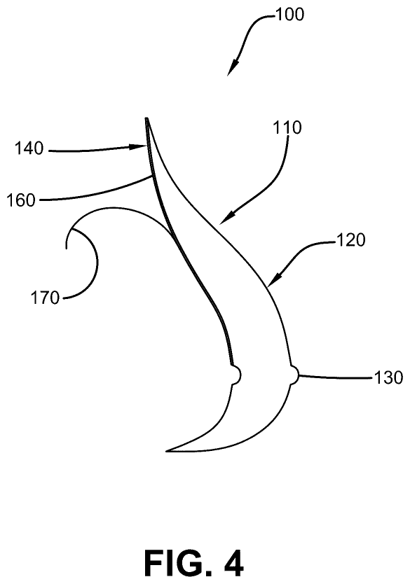

As illustrated in FIGS. 3 and 4, the breast onlay mold 100 comprises a body 110. The body 110 is typically manufactured, three dimensionally printed, or molded from a material is mechanically similar to breast tissue. The body 110 may be an additively manufactured body. The body 110 is typically manufactured from silicone, but may be manufactured from thermoplastic elastomer, neoprene, rubber, polymers, or any similar material that may realistically simulate breast tissue. The body 110 may typically have a volume of between 100 cc and 500 cc, although it may be smaller or larger as needed for a particular patient.

The body 110 comprises an outward facing side 120 and an inward facing side 140. The outward facing side 120 is configured to approximate an ideal augmented surface topography of an augmented breast in the physical world. The outward facing side 120 comprises a nipple 130. The nipple 130 extends outward from the outward facing side 120. The nipple 130 may be customizable or adjustable.

The inward facing side 140 comprises a negative mold 150 of a native breast. The negative mold 150 of the native breast is configured to approximate a surface topography of the native breast. The negative mold 150 is customized to mimic a patient's existing chest wall and breast anatomy.

The breast onlay mold 100 further comprises an adhesive layer 160. The adhesive layer 160 is applied to the inward facing side 140 of the body 110. The adhesive layer 160 is designed to adhere the breast onlay mold 100 to a patient's existing upper torso and breast anatomy. The adhesive layer 160 is ideally a hypoallergenic adhesive. The adhesive layer 160 may be a silicone based adhesive configured to temporarily secure the body 110 over the native breast. The adhesive layer 160 may alternatively be a Derma-tac™ adhesive, a Dermabond® adhesive, a SurgiSeal™ adhesive, a water-based adhesive, an adhesive tape, or any other temporary skin-safe adhesive.

The breast onlay mold 100 may further comprise a release layer 170. The release layer 170 temporarily covers and protects the adhesive layer 160 prior to application. The release layer 170 simply peels off of the breast onlay mold 100 to expose the adhesive layer 160. The breast onlay mold 100 may further comprise a variety of skin tones or colors to mimic a realistic appearance of a variety of people. A color of the body 110 may be configured to match a skin tone of the patient.

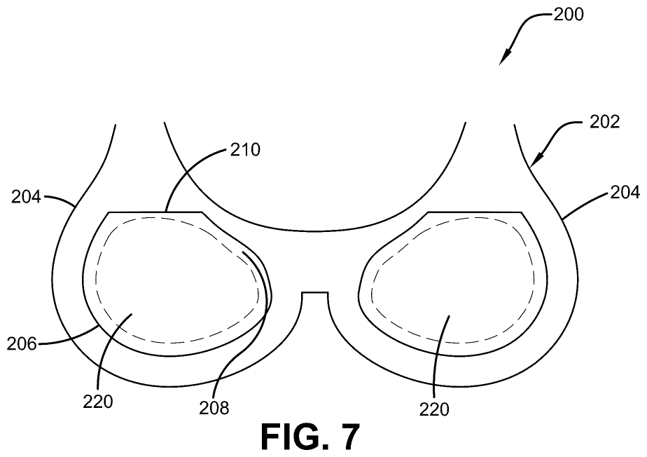

The breast onlay mold 100 may be configured to act as a physical simulation of a breast lift for mastopexy, where the breast onlay mold 100 is configured as a volume neutral on-plant as illustrated in FIG. 6 for reshaping a human breast without adding additional volume. The breast onlay mold 100 is alternatively configured to act as a physical simulation of a breast augmentation, where the breast onlay mold 100 is configured as a volume positive on-plant as illustrated in FIG. 7 for reshaping a human breast by adding additional volume.

The main difference between the breast onlay mold 100 and exiting options is that the breast onlay mold 100 approximates the surface topography of the augmented or reconstructed breast in the physical world, not simply the volumetric change in the physical world or the topographical change in the virtual world. The breast onlay mold 100 is a soft 3D printed mold that is customized to a patient's own chest and breast geometry. Unlike other simulation tools, the present invention is designed image to the patient's own breast and chest geometry using any of the available 3D scanning technologies that are widely available. The breast onlay mold 100 adds precise volume to the patient's native breast in an anatomically relevant manner.

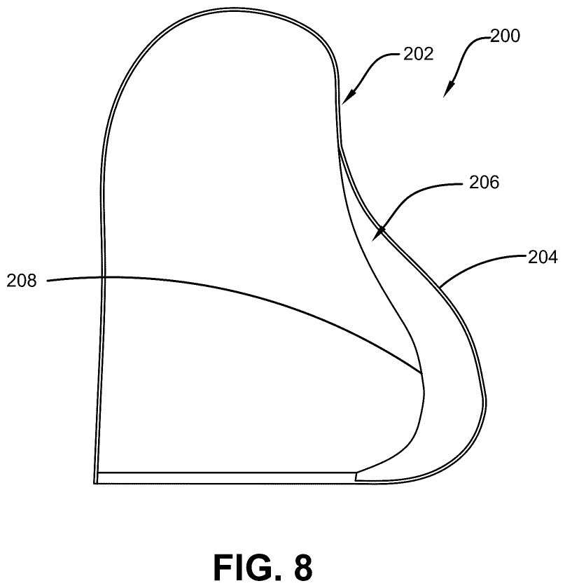

The subject matter disclosed and claimed herein, in an additional embodiment thereof, comprises a breast augmentation simulating apparatus 200. As illustrated in FIGS. 7 and 8, the breast augmentation simulating apparatus 200 comprises a brassiere 202 and a pair of breast onlay molds 220. The brassiere 202 comprises a pair of cups 204. Each cup 204 comprises a pocket 206. Each pocket 202 is configured to retain one of the breast onlay molds 220 within. Each pocket 202 may be formed in a front side or a back side of the respective cup 204 and may have a closure 210, such as a zipper, an elastic closure, a snap, a button, a magnet, a hook and loop fastener, or the like. Each pocket 202 may comprise a negative mold 208 of a native breast.

Similar to the breast onlay mold 100 of the previous embodiment, each breast onlay mold 220 comprises a body 110. Each body 110 is typically manufactured, three dimensionally printed, or molded from a material is mechanically similar to breast tissue. Each body 110 may be an additively manufactured body. Each body 110 is typically manufactured from silicone, but may be manufactured from thermoplastic elastomer, neoprene, rubber, polymers, or any similar material that may realistically simulate breast tissue. Each body 110 may typically have a volume of between 100 cc and 500 cc, although it may be smaller or larger as needed for a particular patient. Each body 110 may be of a different size and surface topography.

Each body 110 comprises an outward facing side 120 and an inward facing side 140. Each outward facing side 120 is configured to approximate an ideal augmented surface topography of an augmented breast in the physical world. Each outward facing side 120 comprises a nipple 130. Each nipple 130 extends outward from the outward facing side 120. Each nipple 130 may be customizable or adjustable.

Each inward facing side 140 comprises a negative mold 150 of a native breast. Each negative mold 150 of the native breast is configured to approximate a surface topography of the native breast. Each negative mold 150 is customized to mimic a patient's existing chest wall and breast anatomy.

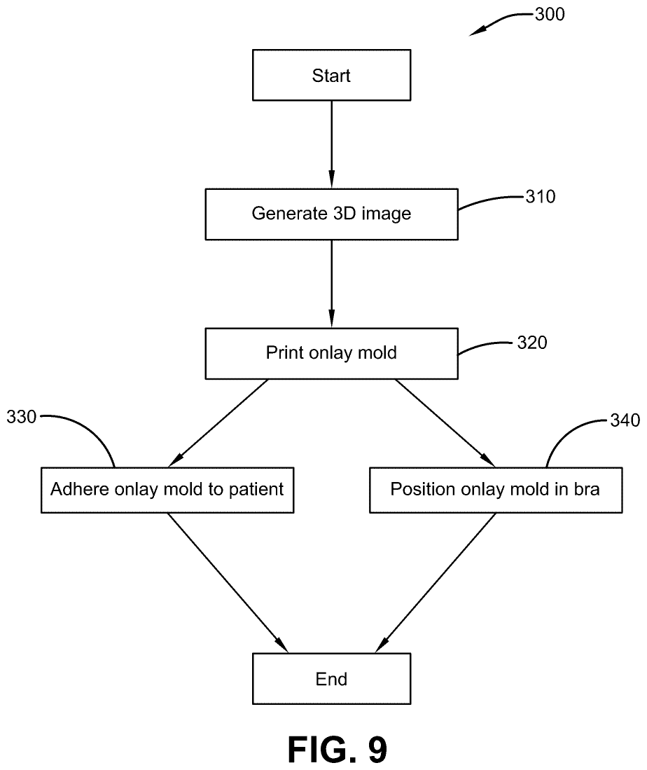

The subject matter disclosed and claimed herein, in an additional embodiment thereof, comprises a method 300 of simulating a breast augmentation on a person for use in the physical world. As illustrated in FIG. 9, the method 300 begins at 310 by obtaining a three dimensional image of the patient's upper front torso and breasts. Next, at 320 a breast onlay mold 100 is generated. The breast onlay mold 100 may be additively manufactured or molded in a soft material. The breast onlay mold 100 may be manufactured in customizable volumes, shapes, and weights.

The breast onlay mold 100 comprises an outward facing side 120 and an inward facing side 140. The outward facing side 120 is configured to approximate an ideal augmented surface topography of an augmented breast in the physical world. The outward facing side 120 comprises a nipple 130. The nipple 130 extends outward from the outward facing side 120. The nipple 130 may be customizable or adjustable.

The inward facing side 140 comprises a negative mold 150 of a native breast. The negative mold 150 of the native breast is configured to approximate a surface topography of the native breast. The negative mold 150 is customized to mimic a patient's existing chest wall and breast anatomy.

Once manufactured, the breast onlay mold 100 is adhered to a torso over a native breast at 330. A release layer 170 may be peeled off of an adhesive layer 160 to expose the adhesive. Alternatively, at 340 the breast onlay mold 100 may be positioned in a special brassiere 202 as described supra, and positioned over the patient's chest to provide the best possible approximation of augmentation, mimicking both the volume change and the effect it will have on the skin.

The patient receives a soft-to-the-touch overlay that attaches by skin adhesive to her chest wall or sits in a specially designed bra that contains a negative mold of her native breast with the addition of an amount of volume chosen by the patient. For example, if her native breast has a volume of 300 cc and she wishes to have a 500 cc breast, the breast onlay mold 100 would contain 200 cc of anatomically relevant volume oriented like her 500 cc augmented breast. The present invention allows a surgeon to help explain an operation to a patient. Additionally, present invention provides an opportunity for patients to explore different options on their own, to “try-on” a lift or augmentation, and to live with a potential change for hours to days

It is contemplated that the breast onlay mold 100 and the breast augmentation simulating apparatus 200 constructed in accordance with the present invention will be tailored and adjusted by those of ordinary skill in the art to accommodate various levels of performance demand imparted during actual use. Accordingly, while this invention has been described by reference to certain specific embodiments and examples, it will be understood that this invention is capable of further modifications. This application is, therefore, intended to cover any variations, uses or adaptations of the invention following the general principles thereof, and including such departures from the present disclosure as come within known or customary practice in the art to which this invention pertains and fall within the limits of the appended claims.

Notwithstanding the foregoing, the breast onlay mold 100 and the breast augmentation simulating apparatus 200 of the present invention and its various structural components can be of any suitable size, shape, and configuration as is known in the art without affecting the overall concept of the invention, provided that it accomplishes the above stated objectives. One of ordinary skill in the art will appreciate that the breast onlay mold 100 and the breast augmentation simulating apparatus 200 and its various components and material, as shown in the FIGS. are for illustrative purposes only, and that many other shapes and sizes of the breast onlay mold 100 and the breast augmentation simulating apparatus 200 are well within the scope of the present disclosure. Although the dimensions of the breast onlay mold 100 and the breast augmentation simulating apparatus 200 are important design parameters, the breast onlay mold 100 and the breast augmentation simulating apparatus 200 and its components may be of any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A breast onlay mold comprising:
a body comprising an outward facing side and an inward facing side; and
an adhesive layer applied to the inward facing side; and
wherein the inward facing side comprises a negative mold of a native breast.

2. The breast onlay mold of claim 1 further comprising a release liner covering the adhesive layer.

3. The breast onlay mold of claim 1, wherein the outward facing side comprises a nipple.

4. The breast onlay mold of claim 3, wherein the nipple is adjustable on the outward facing side.

5. The breast onlay mold of claim 1, wherein the body is an additive manufactured body.

6. The breast onlay mold of claim 1, wherein the body is a silicon body.

7. The breast onlay mold of claim 1, wherein the body is a thermoplastic elastomer body, a neoprene body, or a rubber body.

8. The breast onlay mold of claim 1, wherein the negative mold is configured to approximate a surface topography of a native breast.

9. The breast onlay mold of claim 1, wherein the body has a volume of between 100 cc and 500 cc.

10. The breast onlay mold of claim 1, wherein the adhesive layer comprises a silicone adhesive.

11. The breast onlay mold of claim 1, wherein the adhesive layer comprises an adhesive tape.

12. The breast onlay mold of claim 1, wherein the adhesive layer is hypoallergenic.

13. The breast onlay mold of claim 1, wherein a color of the body is configured to match a skin tone of a patient.

14. A breast augmentation simulating apparatus comprising:
a brassiere comprising a pair of cups, wherein each cup comprises a pocket;
a pair of breast onlay molds, wherein each breast onlay mold comprises a body comprising an outward facing side and an inward facing side; and
wherein each inward facing side comprises a negative mold of a native breast; and
wherein each breast onlay mold is configured to fit within one of the pockets of the brassier.

15. The breast onlay mold of claim 14, wherein each pocket comprises a negative mold of a native breast.

16. The breast onlay mold of claim 14, wherein each pocket comprises a closure.

17. The breast onlay mold of claim 14, wherein each body is an additive manufactured silicone body.

18. The breast onlay mold of claim 14, wherein each negative mold is configured to approximate a surface topography of a native breast.

19. A method of simulating a breast augmentation on a person comprising the steps of:
obtaining a three dimensional image of an upper torso and existing breasts of a person;
generating a breast onlay mold comprising an outward facing side and an inward facing side comprising a negative mold of a native breast; and
adhering the breast onlay mold to a torso over a native breast.

20. The method of claim 19, wherein the breast onlay mold is additively manufactured.
